(19) United States
(12) Patent Application Publication
(10) Pub. No.: US 2005/0175679 A1
(43) Pub. Date: Aug. 11, 2005

(54) Controlled release formulations

(76) Inventors: Michael Moshman, New Rochelle, NY (US); Fred H. Mermelstein, Upper Montclair, NJ (US)

Correspondence Address: DARBY & DARBY P.C., P. O. BOX 5257, NEW YORK, NY, 10150-5257, US

(21) Appl. No.: 10/776,333

(22) Filed: Feb. 10, 2004

(51) Int. Cl. A61K 9/70; A61K 9/00; A61K 31/485

(52) U.S. Cl. 424/449; 514/282; 514/55

(57) ABSTRACT

The present invention relates to controlled release transmucosal formulations which mediate absorption and methods of use comprising a pharmaceutically active agent, preferably morphine, and a water soluble polymer, chitosan, and preferably one more antioxidants, one or more antimicrobial agents, and water.

### FIELD OF THE INVENTION

The present invention relates to controlled release transmucosal formulations which mediates absorption and methods of their use. More particularly, the invention relates to compositions comprising a pharmaceutically active ingredient, e.g., morphine, and a chitosan polymer.

### BACKGROUND OF THE INVENTION

Sustained release dosage forms are central in the search for improved therapy, both through improved patient compliance and decreased incidences of adverse drug reactions. The challenge is to administer a single dose of the drug which is sufficient to maintain the desired concentration over a prolonged period, while eliminating the possibility of overdosing at the outset. In the case of transmucosal administration, controlled release has been difficult to impart, because, in contrast to oral dosage forms, it is not feasible to coat or otherwise compound the drug so that the delivery of the drug is retarded in the body after administration. Longer periods of response provide for many therapeutic benefits that are not achieved with corresponding short acting, immediate release preparations. Thus, therapy may be continued without interrupting the sleep of the patient, which is of special importance, for example, when treating a patient for moderate to severe pain (e.g., a post-surgery patient, a cancer patient, etc.), or for those patients who experience migraine headaches on awakening, as well as for the debilitated patient for whom sleep is essential. A further general advantage of longer acting drug preparations is improved patient compliance resulting from the avoidance of missed doses through patient forgetfulness.

Without a means of controlling release, rapid acting drug therapy requires careful administration at frequent intervals to maintain effective steady state blood levels of the drug, and to avoid peaks and valleys in the blood level because of the rapid absorption, and systemic excretion of the compound through metabolic inactivation. These peaks and valleys cause special problems in maintenance therapy of the patient. In view of this, it is considered a goal that a controlled release dosage form will ideally provide therapeutic concentration of the drug in blood that is maintained throughout an extended dosing interval with a reduction in the peak/trough concentration ratio. Central to the development process are the many variables that influence the in vivo release and subsequent absorption of the active ingredients.

Therefore, there remains a need in the art for additional opioid salts capable of use in compositions directed to controlled release administration by transmucosal delivery, particularly for nasal administration.

### SUMMARY OF THE INVENTION

According to the invention, it has been discovered that transmucosal compositions comprising a highly concentrated pharmaceutically active agent, preferably morphine, and a water soluble polymer, namely chitosan, will mediate absorption of the active agent after administration. The ratio of the two components at specific concentrations achieves optimum controlled release performance.

These and other aspects of the invention are discussed more in the detailed description and examples.

### DESCRIPTION OF THE DRAWINGS

FIG. 1 presents morphine plasma concentration (ng/ml) over time (minutes) for a 15 mg morphine composition with chitosan (indicated by triangle) and a 15 mg morphine composition without chitosan (indicated with circle).

FIG. 2 presents the following mean plasma concentration-time profiles of morphine (ng/ml over hours) formulations with chitosan: 10 mg intravenous morphine formulation, intranasal morphine formulations (7.5 mg, 15 mg and 30 mg), and 15 mg oral morphine formulation.

FIG. 3 presents the mean (±SD) plasma concentration-time profiles of morphine (ng/ml over hours) following intranasal morphine formulations (7.5 mg, 15 mg and 30 mg) and 10 mg intravenous morphine plus intranasal placebo.

FIG. 4 presents the mean (±SD) plasma concentration time profiles of morphine-6-glucuronide (ng/ml over hours) following intranasal morphine formulations (7.5 mg, 15 mg and 30 mg) and 10 mg intravenous morphine plus intranasal placebo.

FIG. 5 presents the mean (±SD) plasma concentration time profiles of morphine-3-glucuronide (ng/ml over hours) following intranasal morphine formulations (7.5 mg, 15 mg and 30 mg) and 10 mg intravenous morphine plus intranasal placebo.

FIG. 6 presents the linear relationship between the bioavailability of intranasal morphine (represented as area under the curve in ng/ml/min.) and the administered dose (in mg).

### DETAILED DESCRIPTION OF THE INVENTION

Compositions of the present invention contain a therapeutically effective amount of at least one pharmaceutically acceptable medicament (active ingredient). Possible pharmaceutically active ingredients include but are not limited to analgesics, anesthetics, decongestants, hypnotics, sedatives, antiepileptics, awakening agents, psychoneurotropic agents, neuromuscular blocking agents, antispasmodic agents, antihistaminics, antiallergics, cardiotonics, antiarrhythmics, diuretics, hypotensives, vasopressors, antitussive expectorants, thyroid hormones, sexual hormones, antidiabetics, antitumor agents, antibiotics, chemotherapeutics, and other CNS acting agents.

In a preferred embodiment, the pharmaceutically active ingredient is an opioid. As used herein the term “opioid” means all agonists and antagonists of opioid receptors, such as mu, kappa, and delta opioid receptors and subtypes thereof. For a discussion of opioid receptors and subtypes, see Goodman and Gilman's The Pharmacological Basis of Therapeutics 9th ed. J. G. Harman and L. E. Limird Eds., McGraw-Hill New York:1996 pp. 521-555, incorporated herein by reference. Preferred opioids interact with the mu opioid receptor, the kappa opioid receptor, or both. Preferably, the opioid is an opioid-receptor agonist. Illustrative categories and specific examples of opioids include, but are not limited to, high potency analgesics (where specific salts or esters are mentioned, it should be understood to include other salt, ester, or free acid forms of the drug), such as fentanyl, codeine, or morphine.

In the preferred embodiment, the opioid is morphine. The morphine compound may be selected from, but are not limited to, one of the following compounds: morphine base monohydrate, morphine hydrochloride, morphine sulfate, morphine mesylate, morphine citrate, morphine ascorbate and other salts of morphine. Preferably, the morphine is purified morphine base monohydrate (anhydrous base, MW 303.36), C₁₇H₁₉O₃N.H₂O, having the following structural formula: 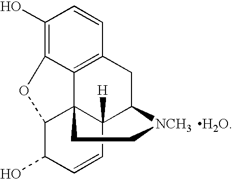
Morphine base (purified, monohydrate) is preferred since it binds to the opiate receptors with higher affinity and is a strong agonist.

Depending on the opioid compound, the composition will vary, however, the medicament may be present in the composition from about 18.75 mg/ml to about 300 mg/ml, preferably from about 37.5 mg/ml to about 150 mg/ml. Most preferred, the medicament is present in an amount of about 75 mg/ml.

Various pharmaceutically acceptable salts, ether derivatives, ester derivatives, acid derivatives, and aqueous solubility altering derivatives of the active compound also are encompassed by the present invention. The present invention further includes all individual enantiomers, diastereomers, racemates, and other isomer ratios of the compound. The invention also includes all polymorphs and solvates, such as hydrates and those formed with organic solvents, of this compound. Such isomers, polymorphs, and solvates may be prepared by methods known in the art, such as by regiospecific and/or enantioselective synthesis and resolution, based on the disclosure provided herein.

Suitable salts of the compound include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compound of the present invention; acid addition salts including but not limited to salts made with saccharin; alkali metal salts; alkaline earth metal salts; and salts formed with organic or inorganic ligands. Preferably, the morphine salt is a morphine mesylate salt.

The present invention also includes prodrugs of the compound of the present invention. Prodrugs include, but are not limited to, functional derivatives of the pharmaceutically active agents that are readily convertible in vivo into the target agents. Conventional procedures for the selection and preparation of suitable prodrug derivatives are described, for example, in “Design of Prodrugs” (ed. H. Bundgaard, Elsevier, 1985).

The controlled release material, in effect, acts as a carrier for the active agent. The preferred polymer in the present invention is Chitosan ([(1,4)-2-amino-2-desoxy-b-D-glucan]), a commercially available, nontoxic polymer or a salt or derivative thereof. Chitosan is a linear polysaccharide derived from the shells of crustaceans. The material can further include a bioadhesive or mucoadhesive polymer such as pectins (polygalacturonic acid), mucopolysaccharides (hyaluronic acid, mucin) or non-toxic lectins. The polymer itself may be bioadhesive, e.g., polyanhydride or polysaccharides such as chitosan.

As used herein, “chitosan” includes all derivatives of chitin, e.g., poly-N-acetyl-D-glucosamine, including all polyglucosamines and oligomers of glucosamine materials of different molecular weights, in which the greater proportion of the N-acetyl groups have been removed through hydrolysis (deacetylation). Preferably, the chitosan is produced from chitin by deacetylation to a degree of greater than 40%, preferably about 50% to 98%, and more preferably about 70% to 90%. Chitosan derivatives or salts of chitosan (e.g., nitrate, phosphate, sulphate, hydrochloride, glutamate, lactate or acetate salts) may also be used instead of chitosan. As used herein, “chitosan derivatives” includes ester, ether or other derivatives formed by bonding of acyl and/or alkyl groups with OH groups, but not the NH₂ groups, of chitosan. Examples include O-alkyl ethers of chitosan and O-acyl esters of chitosan. Modified chitosans, particularly those conjugated to polyethylene glycol, are included in this definition. Low and medium viscosity chitosans (for example, CL113, G210 and CL110) may be obtained from various sources, including Pronova Biopolymer (Drammen, Norway); Seigagaku America Inc., (MD, USA); Meron Pvt, Ltd. (India); Vanson Ltd, (VA, USA); and AMS Biotechnology Ltd., (UK). Suitable derivatives include those which are disclosed in Roberts, Chitin Chemistry, (MacMillan Press Ltd., London (1992)).

The chitosan, chitosan derivative or salt, of the present invention preferably has a molecular weight of about 4,000 Daltons or more, preferably in the range of about 25,000 to about 2,000,000 Daltons, and most preferably in the range of about 250,000 to about 600,000 Daltons.

Chitosans of different low molecular weights can be prepared by enzymatic degradation of chitosan using chitosanase or by the addition of nitrous acid. Both procedures are known to those skilled in the art. Preferably, the chitosan compound is water-soluble. Particularly preferred chitosan compounds, which may be mentioned, include the UPG210 and UPG 213 chitosan available from FMC Corporation (Philadelphia, Pa.). UPG210 and UPG 213 chitosan are high molecular weight range materials that are highly purified thereby allowing for controlled release or more regularized bioavailability and are therefore more appropriate for the consistency of delivery of a pharmaceutical grade material.

In the present invention, the ratio of the pharmaceutically active ingredient to the chitosan polymer must be within a specific range to obtain the controlled release properties of the chitosan polymer. The ratio will vary depending on the molecular weight of the compounds used, for example, depending on the specific chitosan used. Therefore, in the present invention, the ratio is preferably calculated on the basis of a molecule to molecule ratio. The molecule to molecule ratio of the active ingredient to the chitosan may be from about 1:1 to about 100,000:1, preferably, from about 5,000:1 to about 80,000:1.

Alternatively, for convenience, where the specific compounds are known, the ratio of the chitosan and active ingredient may be expressed on weight to weight or weight to volume basis. For example, in a preferred embodiment of the present invention, purified morphine base monohydrate (molecular weight 303.4) is combined with the preferred chitosan (having a molecular weight of approximately 420,000). In the preferred embodiment, the applicable ratio of morphine to the chitosan described above is from about 5:1 to about 60:1. Preferably, the ratio is from about 7.5:1 to about 30:1. In the present invention, the chitosan polymer may be present in ranges of about 2 mg/ml to about 7 mg/ml, preferably about 4 mg/ml to about 6 mg/ml. The most preferred amount in the composition is about 5 mg/ml.

The formulations of the present invention are designed to produce a controlled increase in therapeutic plasma levels of the pharmaceutically active ingredient during the absorption phase after nasal administration. This mediated absorption of the medicament is followed by a period of controlled dissolution of the medicament to maintain therapeutic plasma levels. Without the controlled release during the absorption phase, there is a risk of too rapid absorption when applying the dosage necessary to maintain a therapeutic level of the medicament over a prolonged period. Too rapid absorption may lead to overdosage. The chitosan formulation of the present invention has demonstrated regularized and mediated absorption by first order rate kinetics during the absorption phase of the product when delivered to the nasal mucosa. For example, absorption of morphine formulated without chitosan is non-linear during the uptake phase; however, the same formulation with chitosan demonstrates linear uptake.

The compositions of the present invention may also contain one or more pharmaceutically acceptable antioxidants. Non-limiting examples include methanesulfonic acid, citric acid, sodium citrate, ascorbic acid, and sodium ascorbate.

The total amount of antioxidants present in the composition is from about 20 to 50 mg per ml for the citric acid/sodium citrate formulations and a range of about 20 to about 40 mg per ml to be used as particularly suitable. For example, citric acid may be present in an amount ranging from about 10 to about 20 mg/ml, and the sodium citrate may be present in an amount ranging from about 5 to about 20 mg/ml. For the ascorbic acid/sodium ascorbate formulation, the amount of antioxidants present in the composition is from about 40 to about 70 mg per ml and a particularly suitable range from about 50 to about 65 mg per ml. For example, ascorbic acid may be present in an amount ranging from about 40 to about 50 mg per ml, and sodium ascorbate may be present from about 10 to about 15 mg/ml. For compositions using methanesulfonic acid, the antioxidant is present in the composition from about 10 to about 60 mg per ml, and a particularly suitable range from about 13 to about 50 mg per ml.

The antioxidants of the present invention have a buffering effect and are used in amounts sufficient to adjust and maintain the pH of the compositions of the present invention in the range of about 3.0 to about 7.0, preferably about 4.0 to about 5.0. Typically suitable buffers include, but are not limited to, citrates, ascorbates, phosphates and glycines. Citrate and ascorbate are excellent antioxidants and therefore protect the morphine molecule from oxidative degradation and therefore improve the overall stability of the formulation. Furthermore, both citrate and ascorbate are good buffering agents and therefore allow the drug product to be maintained within a pH range that lends stability (shelf-life) to the morphine containing formulation.

The compositions of the present invention also contain at least one antimicrobial preservative in the range of 0.0005% to about 0.5% by weight/volume of the composition, preferably in the range of 0.005% to 0.5% by weight/volume to accommodate the combination of excipients that can be construed as antimicrobials by weight/volume of the composition. Typical suitable antimicrobial agents include benzalkonium chloride (BAK), benzethonium chloride, disodium EDTA, and sodium benzoate. The range of amounts of antimicrobials used in the present invention are dependent upon the particular components used. For example, a preferred amount of BAK is about 0.15 mg/mL (0.015%). A preferred amount of disodium EDTA is about 11.0 mg/mL (0.1%). A preferred amount of sodium benzoate is about 0.2 mg/mL (0.02%).

The initial amounts of ascorbic acid or citric acid is used to insure solubility of the morphine. In addition, a combination of the acid and sodium salts of the acid will be used to adjust the pH of the resultant solution to between 4.0 and 4.5. Both acids are excellent antioxidants and produce a significant improvement over the existing formulation. The sodium EDTA is used primarily as a chelating agent, and with either BAK or sodium benzoate are used for the antimicrobial capability of these combinations.

As used herein the term “transmucosal” refers to the mode of administration of the formulation. The transmucosal modes of administration include, but are not limited to, nasal, buccal, rectal, vaginal, and occular modes of administration. Preferably, the formulation is administered nasally.

The term “amount” as used herein refers to quantity or to concentration as appropriate to the context. The amount of a drug that constitutes a therapeutically effective amount varies according to factors such as the potency of the particular drug, the route of administration of the formulation, and the mechanical system used to administer the formulation. A therapeutically effective amount of a particular drug can be selected by those of ordinary skill in the art with due consideration of such factors.

The phrase “pharmaceutically acceptable” refers to molecular entities and compositions that are “generally regarded as safe”, e.g., that are physiologically tolerable and do not typically produce an allergic or similar untoward reaction, such as dizziness and the like, when administered to a human. Preferably, as used herein, the term “pharmaceutically acceptable” means approved by a regulatory agency of the Federal or a state government or listed in the U.S. Pharmacopeia or other generally recognized pharmacopoeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Company, Easton, Pa., USA 1985).

### Morphine Formulations

The compositions of the present invention are manufactured in a conventional manner such as by mixing the ingredients under nitrogen gas at ambient or elevated temperatures to achieve solubility of ingredients where appropriate. Specifically, the solution may be prepared as follows.

To any appropriate reaction container, the active agent and the acid solution are mixed together. The polymer and antimicrobial agent are mixed together. The two mixtures are combined and chelating agents are mixed together. Each ingredient is mixed until the solution appears homogenous. The antioxidants and buffers are added to the mixture to adjust the pH of the solution. The final batch volume is adjusted with any suitable liquid, e.g., water. The solution is further mixed until uniform and filtered with a pre-sterilized filter using conventional filtration equipment. Preferably, a pre-sterilized 0.22 micron filter is used.

In one embodiment, the solution yields an osmolality of about 200 mOsm to about 900 mOsm. Preferably, the solution yields an osmolality of about 400 to about 600 mOsm. Most preferred, the solution yields an osmolality of about 500 mOsm.

In another embodiment, the viscosity of the solution is from about 1 to about 50 centipoise. It is preferable to have a low viscosity as spray droplet size is small with a lower viscosity product optimizing surface area exposure and more regularized (reliable) delivery of product.

In the present invention, the composition yields about 18.75 to about 300 microgram of pharmaceutically effective agent per 100 microliter nasal spray.

The dosage forms used may be administered alone or in combination with other active agents. For combination treatment with more than one active agent, where the active agents are in separate dosage formulations, the active agents can be administered concurrently, or they each can be administered at separately staggered times. The dosage may be adjusted when combined with other active agents as described above to achieve desired effects. On the other hand, unit dosage forms of these various active agents may be independently optimized.

The present invention will be better understood by reference to the following Examples, which are provided as exemplary of the invention, and not by way of limitation.

### EXAMPLE 1

### Morphine Nasal Spray Formulation

An aqueous nasal spray composition is prepared from the following components:

| Ingredients | Weight/ml |
|---|---|
| Morphine, anhydrous base | 75.0 mg |
| Methanesulfonic acid | 25.3 mg |
| Benzalkonium chloride (BAK) | 0.15 mg |
| Edatate Disodium, USP | 1.0 mg |
| Chitosan | 5 mg |
| WFI Water | QS to 1 ml |

To any appropriate reaction container, the active agent and the methanesulfonic acid solution are mixed together. The polymer and antimicrobial agent are mixed together. The two mixtures are combined and chelating agents are mixed together. Each ingredient is mixed until the solution appears homogenous. The antioxidants and buffers are added to the mixture to adjust the pH of the solution. The final batch volume is adjusted with any suitable liquid, e.g., water. The solution is further mixed until uniform, with a pH value ranging between 3.0-5.0, and filtered with a pre-sterilized filter using conventional filtration equipment. Preferably, a pre-sterilized 0.22 micron filter is used.

The solution yields an osmolality of about 500 mOsm. Viscosity of the solution measures less than 50 centipoise. The resulting formulation yields a 7.5 milligram morphine per 100 micro liter spray.

### EXAMPLE 2

### Morphine Nasal Spray Formulation

An aqueous nasal spray composition is prepared from the following components:

| Ingredients | Weight/ml |
|---|---|
| Morphine base (MW 303.4) | 75.0 mg |
| Citric acid (MW 192.12) | 15.9 mg |
| Sodium citrate | 9.0 mg |
| Sodium benzoate (MW = 144.10) | 0.2 mg |
| Disodium EDTA | 1.0 mg |
| Chitosan | 5.0 mg |
| WFI Water | QS to 1 ml |

To any appropriate reaction container, the active agent and the citric acid solution are mixed together. The polymer and antimicrobial agent are mixed together. As an alternative to sodium benzoate, benzalkonium chloride may be used in an amount of 0.15 mg. The two mixtures are combined and chelating agents are mixed together. Each ingredient is mixed until the solution appears homogenous. The antioxidants and buffers are added to the mixture to adjust the pH of the solution. The final batch volume is adjusted with any suitable liquid, e.g., water. The solution is further mixed until uniform and filtered with a pre-sterilized filter using conventional filtration equipment. Preferably, a pre-sterilized 0.22 micron filter is used. The solution yields an osmolality of about 500 mOsm. Viscosity of the solution measures less than 50 centipoise. The resulting formulation yields a 7.5 milligram morphine per 100 microliter spray.

### EXAMPLE 3

### Morphine Nasal Spray Formulation

An aqueous nasal spray composition is prepared from the following components:

| Ingredients | Weight/ml |
|---|---|
| Morphine base (MW = 303.4) | 75.0 mg |
| Ascorbic acid (MW = 176.12) | 43.5 mg |
| Sodium ascorbate | 12.0 mg |
| BAK | 0.15 mg |
| Disodium EDTA | 1.0 mg |
| Chitosan | 5.0 mg |
| WFI Water | QS to 1 ml |

The solution is prepared as follows. To any appropriate reaction container, the active agent and the ascorbic acid solution are mixed together. The polymer and antimicrobial agent are mixed together. The two mixtures are combined and chelating agents are mixed together. Each ingredient is mixed until the solution appears homogenous. The antioxidants and buffers are added to the mixture to adjust the pH of the solution. The final batch volume is adjusted with any suitable liquid, e.g., water. The solution is further mixed until uniform and filtered with a pre-sterilized filter using conventional filtration equipment. Preferably, a pre-sterilized 0.22 micron filter is used.

The solution yields an osmolality of about 500 mOsm. Viscosity of the solution measures less than 50 centipoise. The resulting formulation yields a 7.5 milligram morphine per 100 microliter spray.

### EXAMPLE 4

### Process Description of Morphine Formulations

The following exemplifies a method of preparation of the 1 liter batch size for the morphine and chitosan formulation:

The process begins by making stock solutions of citric acid (20 gm in a 200 ml volumetric flask) and sodium citrate (10 gm in a 100 ml volumetric flask) in purified water, USP in slight excess of the amount needed for formulating the batch. In the case of the ascorbic acid formulation, a similar process of making the stock solutions beforehand will be performed. A stock solution of BAK is also made and assayed prior to manufacture to enable an accurate amount of this ingredient to be added to the batch.

600 ml of purified water is added to a mixing vessel and stirred using nitrogen to remove dissolved oxygen. 2 ml of citric acid solution is added to the 600 ml while stirring. 5 gm of chitosan is slowly added to the mixing vessel under constant nitrogen and mixing.

159 ml of the citric acid stock solution is added to a second mixing vessel under constant nitrogen sparging. 79.8 gm of purified morphine base monohydrate is added to the mixing vessel while mixing to dissolve the morphine. 79.8 gm is equivalent to 75 gm of the anhydrous base.

The chitosan solution is quantitatively added to the morphine citrate solution and mixed, still using the nitrogen sparge. The equivalent of 0.15 gm of BAK is added from the stock solution with constant mixing. The 1 gm of disodium edetate is added and mixed until the solution is clear. 75 ml of the sodium citrate is added under constant mixing. The batch is adjusted to a pH of 4.1 using the citric acid or the sodium citrate solutions.

The batch is filtered through a Millipore Durapore 0.22 micron filter and collected in a collection vessel under a nitrogen stream.

In process tests including pH, Osmolality, morphine assay and BAK assay are performed. Pre and post filtration bioburden testing is performed for reference.

The batch is filled using a peristaltic pump into the packaging containers that are continuously sparged with nitrogen. The package containers are sealed, inspected, labeled and packaged as required. The finished product is tested to include appearance, identification, pH, morphine assay, related substances, spray weight delivery, spray assay delivery, droplet size, spray shape and size, BAK assay, net contents, microbial testing, and others based on final package configuration.

### EXAMPLE 5

### Bioavailability of Intranasal Morphine Formulations

To demonstrate the tolerability and pharmacokinetic profile of a novel controlled release nasal morphine solution containing chitosan the solution was administered to healthy volunteers. The example shows “controlled” release ability of the present invention as demonstrated by regularized absorption of the product through the nasal mucosa, and the first order rate kinetics during the absorption phase of the product when delivered to the nasal mucosa.

### Methods

The study was a randomized, six-way complete crossover trial of single-dose administration of morphine via intranasal, oral and intravenous routes. Each two consecutive treatments were separated by a washout period of at least 3 days. Intranasal formulations were administered double-blind with respect to dose, with oral and intravenous formulations administered in an open label manner. In addition to the test drugs, each limb of the study was performed under a naltrexone block. The opioid antagonist was administered before each study treatment to prevent the centrally mediated effects of morphine and unpleasant effects of opiate administration in naïve subjects.

An aqueous nasal spray composition was prepared from the following components:

| Formula concentration: | Conc. 1 | Conc. 2 | Conc. 3 |
|---|---|---|---|
| Ingredients | Weight/ml | Weight/ml | Weight/ml |
| Morphine, anhydrous base | 37.5 mg | 75.0 mg | 150 mg |
| Methanesulfonic acid | 12.7 mg | 25.3 mg | 50.6 mg |
| Benzalkonium chloride | 0.15 mg | 0.15 mg | 0.15 mg |
| (BAK) | | | |
| Edetate Disodium, USP | 1.0 mg | 1.0 mg | 1.0 mg |
| Chitosan | 5.0 mg | 5.0 mg | 5.0 mg |
| WFI Water | QS to 1 ml | QS to 1 ml | QS to 1 ml |
| Molecule Ratio of | ˜11,500:1 | ˜23,000:1 | ˜46,000:1 |
| Morphine:Chitosan | | | |

The six treatment limbs were as follows:
1. Intranasal morphine base formulation 7.5 mg (3.75 mg per nostril)
2. Intranasal morphine base formulation 15 mg (7.5 mg per nostril)
3. Intranasal morphine base formulation 30 mg (15 mg per nostril)
4. Intranasal morphine base 15 mg (7.5 mg per nostril, contains no chitosan)
5. Oral morphine sulphate (15 mg Oramorph® solution) plus intranasal placebo
6. Intravenous morphine sulphate 10 mg over 30 minutes plus intranasal placebo.
   The subjects received single administration of six morphine treatments. Nasal placebo was administered to volunteers concomitant with intravenous or oral dosages. Intravenous and oral dosage arms were open label. The pharmacodynamic effects of morphine were avoided with naltrexone pre-treatment.

Thirteen subjects (6 male and 7 female) were randomized into the study, of which five males and seven females successfully completed the study. One subject withdrew consent following the completion of two study sessions and was subsequently replaced. Healthy male or female volunteers aged between 18 and 50 years of age. Overtly healthy as determined by medical assessment including: medical history, physical examination, vital signs, ECG and laboratory analysis (haematology, blood chemistry, virology, urinalysis).

Safety, tolerability, pharmacokinetic and statistical evaluations were conducted, as detailed below. Efficacy was not measured as part of this study. Nasal tolerability, clinical laboratory safety data, vital signs, ECG recordings and physical examinations were assessed.

Blood sampling was conducted over 24 hours for pharmacokinetic and metabolite analysis. Nasal tolerability was evaluated by questionnaires and observations. Plasma levels of morphine and its metabolites, morphine-3-glucuronide (M-3-G) and morphine-6-glucuronide (M-6-G) were determined using standard and validated chromatographic methods. Standard model independent pharmacokinetic methods were used to calculate Cmax, tmax, AUC, Fabs and Frel on the basis of plasma morphine, M-3-G and M-6-G levels. Intra-formulation and dose proportionality were also assessed.

Prior to statistical analysis the parameters AUC, AUCt and Cmax were normalized to a 30 mg dose and log transformed. An initial analysis of variance was performed, which included the factors, subject, period, treatment and first order carry-over in the model. As first order carry-over was found not to be statistically significant it was subsequently dropped from the model. The following comparisons were carried out for morphine, M-6-G and M-3-G using the estimate statement in SAS: Dose proportionality, comparison of the formulation with intravenous morphine sulphate, the formulation without chitosan, and oral morphine sulphate treatments.

### Results

Safety and Tolerability. There were no deaths or serious adverse events. No subject withdrew from the study for study drug related reasons. There were no clinically significant abnormal results as assessed by vital sign, ECG, clinical laboratory parameters and physical examination. Nasal tolerability of intranasal administrations was generally good. There were a total of 87 adverse events reported by a total of 13 subjects, 80 of which were treatment emergent, reported by 13 subjects. The most common treatment emergent adverse events reported during the study were headache (16), vomiting (10) and nausea (10).

Pharmacokinetics. The pharmacokinetic profile of morphine alone and morphine with chitosan delivered by the intranasal route is similar to that of morphine delivered by intravenous administration as indicated in Table 1. Pharmacokinetic parameters of morphine in plasma are summarized below:

TABLE 1

| | Morphine | | | | | | |
|---|---|---|---|---|---|---|---|
| | | | | | | 15 mg | 10 mg |
| | | 7.5 mg | 15 mg | 30 mg | 15 mg (no | Oral + IN | IV + IN |
| Parameter | | w/chitosan | w/chitosan | w/chitosan | chitosan) | Placebo | Placebo |
| Cₘₐₓ | Mean ± SD | 33 ± 9 | 67 ± 21 | 62 ± 17 | 26 ± 7 | 25 ± 13 | 70 ± 20 |
| (ng/ml) | % CV | 28.4 | 30.9 | 26.3 | 27.6 | 53.4 | 27.7 |
| | Range | (17-55) | (40-108) | (30-93) | (17-45) | (11-61) | (45-112) |
| tₘₐₓ | Median | 0.2 | 0.3 | 0.2 | 0.5 | 0.5 | 0.5 |
| (h) | Range | (0.2, 0.3) | (0.2-0.3) | (0.2-0.5) | (0.2-1.0) | (0.2-1.0) | (0.3-0.6) |
| AUCₜ | Mean ± SD | 44 ± 14 | 77 ± 19 | 130 ± 34 | 70 ± 36 | 36 ± 21 | 70 ± 23 |
| (ng.h/ml) | % CV | 32.9 | 24.7 | 26.4 | 51.7 | 57.9 | 32.1 |
| | Range | (21-66) | (48-111) | (81-215) | (29-140) | (14.1-81.8) | (41.3-119.9) |
| AUC | Mean ± SD | 49 ± 14 | 84 ± 20 | 139 ± 34 | 71 ± 31 | 45 ± 23 | 75 ± 22 |
| (ng.h/ml) | % CV | 29.1 | 24.3 | 24.4 | 43.7 | 51.8 | 29.2 |
| | Range | (32-74) | (54-113) | (94-218) | (32-124.) | (17.4-87.5) | (47.9-123.2) |
| t_{1/2} | Mean ± SD | 1.9 ± 0.8 | 1.7 ± 0.7 | 2.3 ± 1.0 | 2.2 ± 1.0 | 1.6 ± 0.5 | 1.7 ± 0.5 |
| (h) | % CV | 41.1 | 39.4 | 44.9 | 46.0 | 33.2 | 30.8 |
| | Range | (0.9-3.4) | (1.0-2.9) | (1.1-4.5) | (1.0-4.6) | (0.6-2.4) | (0.9-2.4) |

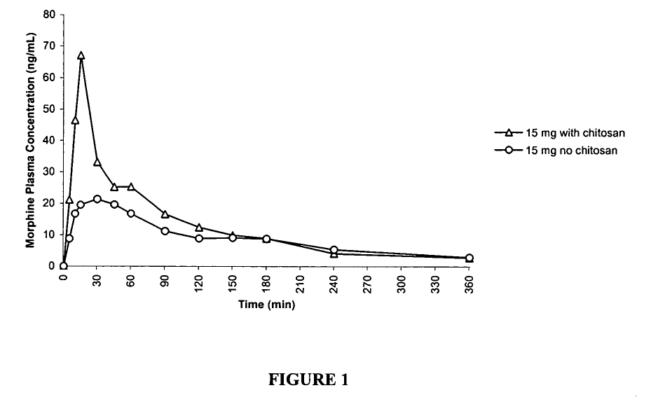

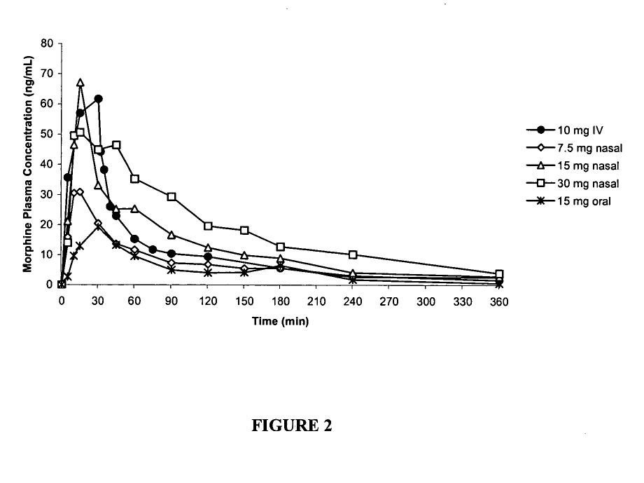

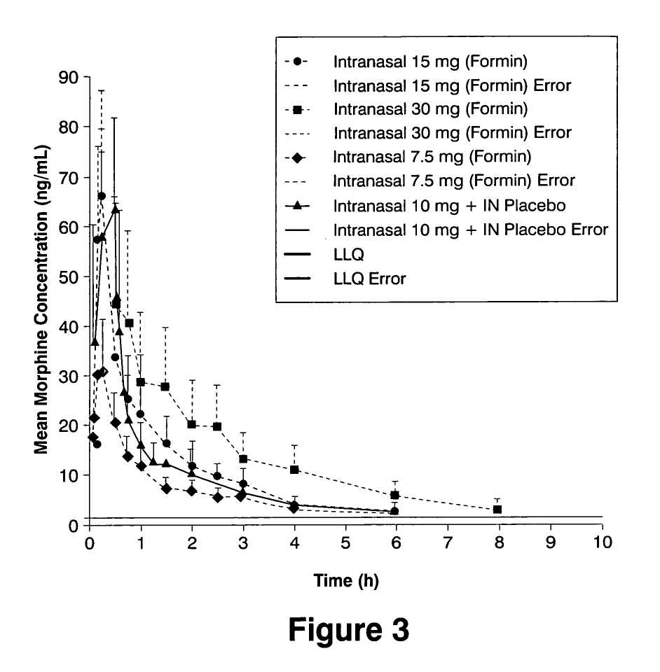

Absorption of morphine formulated without chitosan was non-linear during the absorption phase, whereas first-order rate kinetics is represented for the formulations containing chitosan by linear curves in FIGS. 1 and 2. Linearity is apparent independent of dose of morphine (7.5, 15, 30 mg). This demonstrates controlled absorption. FIG. 3 shows the comparative plasma concentrations of morphine following nasal, oral and intravenous administration.

Based on the 95% CI criteria, dose proportionality could not be concluded for morphine using Cₘₐₓ, AUCₜ and AUC for the intranasal formulation. Statistical analysis revealed the absolute bioavailability of intranasal morphine treatments to be (Geometric means) 82.3%, 95% CI [62.4, 108.5], 74.9%, 95% CI [57.4, 97.6] and 60.4%, 95% CI [46.3, 78.7], for doses of 7.5 mg, 15 mg and 30 mg, respectively. The formulation bioavailability based on statistical analysis for each dose when compared to morphine alone (contains no chitosan) was found to be 139.8%, 95% CI [105.1, 185.9], 127.1%, 95% CI [97.1, 166.5] and 102.5%, 95% CI [78.1, 134.6] for doses of 7.5 mg, 15 mg and 30 mg of the formulation, respectively. Bioavailability was inversely related to dose indiating the greatest effect of the chitosan enhancer to be at lower doses. All intranasal treatments were found to have approximately twice the bioavailability of oral morphine sulphate. Statistically significantly higher Cₘₐₓ values were obtained from the 7.5 mg and 15 mg doses of the formulation when compared with the intranasal morphine base (contains no chitosan). Median tₘₐₓ times were observed to be slightly shorter for the formulations compared to other treatments. Mean values of elimination half-life were comparable between all treatments at approximately 2 hours.

The Pharmacokinetic parameters of morphine-6-glucuronide in plasma are summarized below:

TABLE 2

| | Morphine-6-Glucuronide | | | | | | |
|---|---|---|---|---|---|---|---|
| | | | | | 15 mg | 15 mg | 10 mg |
| | | 7.5 mg | 15 mg | 30 mg | (no | Oral + IN | IV + IN |
| Parameter | | w/chitosan | w/chitosan | w/chitosan | chitosan) | Placebo | Placebo |
| Cₘₐₓ | Mean ± SD | 33 ± 19 | 69 ± 27 | 110 ± 46 | 69 ± 41 | 82 ± 23 | 37 ± 9 |
| (ng/ml) | % CV | 56.6 | 38.5 | 41.9 | 59.0 | 28.0 | 23.9 |
| | Range | (13-80) | (29-116) | (28-191) | (32-192) | (57-128) | (22-53) |
| tₘₐₓ | Median | 1.5 | 1.8 | 1.5 | 1.8 | 1.0 | 1.0 |
| (h) | Range | (0.1, 2.0) | (0.2, 4.0) | (1.0, 2.5) | (1.0, 3.0) | (0.5, 1.5) | (0.7, 2.0) |
| AUCₜ | Mean ± SD | 102 ± 63 | 253 ± 121 | 461 ± 220 | 234 ± 80 | 228 ± 64 | 119 ± 39 |
| (ng.h/ml) | % CV | 61.8 | 47.9 | 47.7 | 34.1 | 27.9 | 32.9 |
| | Range | (14-208) | (79-497) | (99-846) | (128-365) | (157-385) | (61-200) |
| AUC | Mean ± SD | 148 ± 78 | 336 ± 126 | 557 ± 225 | 277 ± 92 | 251 ± 71 | 191 ± 117 |
| (ng.h/ml) | % CV | 52.7 | 37.5 | 40.3 | 33.3 | 28.1 | 61.3 |
| | Range | (70-289) | (187-578) | (166-942) | (177-402) | (168-415) | (102-523) |
| t_{1/2} | Mean ± SD | 3.0 ± 1.6 | 3.6 ± 2.5 | 4.3 ± 2.8 | 3.1 ± 2.1 | 2.0 ± 0.7 | 4.1 ± 4.3 |
| (h) | % CV | 51.4 | 70.0 | 64.6 | 67.4 | 33.6 | 104.2 |
| | Range | (1.3-6.3) | (1.5-9.3) | (1.4-11.1) | (1.3-8.3) | (1.3-3.8) | (2.00-17) |
| AUC | Mean ± SD | 3.0 ± 1.0 | 4.3 ± 2.3 | 4.3 ± 2.0 | 4.3 ± 3.0 | 6.4 ± 2.7 | 2.9 ± 2.5 |
| Metabolic | % CV | 34.8 | 54.2 | 46.4 | 46.1 | 41.2 | 86.9 |
| Ratio¹ | Range | (1.7-4.2) | (1.8-9.3) | (1.8-9.0) | (1.4-7.4) | (3.3-10.9) | (0.9-9.9) |

¹AUC M-6-G/AUC Morphine

Based on the 95% CI criteria dose proportionality could not be concluded for morphine-6-glucuronide using Cₘₐₓ, AUCₜ and AUC for the morphine formulation. Shorter tₘₐₓ ranges and median values for oral and iv treatments, 1.0 (0.5,1.5) h and 1.0 (0.7, 2.0) h respectively, compared to intranasal treatments may indicate more rapid conversion of morphine to morphine-6-glucuronide following these treatments. Mean half-life estimations were quite similar between treatments, ranging between 2.01 h and 4.36 h. The mean half-life time following intravenous morphine sulphate of 4.12 h was distorted due to the value of 16.93 h for subject 10. Mean dose adjusted Cₘₐₓ from the formulations was found to be significantly lower when compared with Cₘₐₓ from the oral formulation with intranasal placebo, this coupled with longer median tₘₐₓ times for intranasal treatments may indicate a longer time for the formation of the metabolite. As expected, the formation of M-6-G from morphine was greatest following oral morphine sulphate due to first pass metabolism and least after intravenous infusion of morphine sulphate. In general the metabolic ratios following the intranasal formulations were comparable, somewhere between the two values for oral and iv infusion.

The Pharmacokinetic parameters of morphine-3-glucuronide in plasma are summarized below:

TABLE 3

| | Morphine-3-Glucuronide | | | | | | |
|---|---|---|---|---|---|---|---|
| | | | | | 15 mg | 15 mg | 10 mg |
| | | 7.5 mg | 15 mg | 30 mg | (no | Oral + IN | IV + IN |
| Parameter | | w/chitosan | w/chitosan | w/chitosan | chitosan) | Placebo | Placebo |
| Cₘₐₓ | Mean ± SD | 159 ± 53 | 342 ± 130 | 543 ± 253 | 394 ± 272 | 454 ± 1034 | 172 ± 38 |
| (ng/ml) | % CV | 33.2 | 37.9 | 46.7 | 69.0 | 22.9 | 22.3 |
| | Range | (94-241) | (186-569) | (195-1084) | (180-1204) | (295-624) | (107-243) |
| tₘₐₓ¹ | Median | 1.5 | 1.5 | 1.5 | 1.5 | 1.0 | 0.9 |
| (h) | Range | (0.8, 3.0) | (1.0, 2.5) | (0.8, 3.0) | (1.0, 3.0) | (0.5, 1.5) | (0.7, 1.3) |
| AUCₜ | Mean ± SD | 796 ± 260 | 1854 ± 494 | 3136 ± 1166 | 1706 ± 556 | 1797 ± 413 | 769 ± 210 |
| (ng.h/ml) | % CV | 32.7 | 26.7 | 37.2 | 32.6 | 23.0 | 27.3 |
| | Range | (528-1294) | (996-2628) | (1230-4774) | (965-2574) | (1468-2684) | (489-1273) |
| AUC | Mean ± SD | 898 ± 318 | 2016 ± 554 | 3510 ± 1273 | 1942 ± 693 | 1948 ± 423 | 871 ± 233 |
| (ng.h/ml) | % CV | 35.4 | 27.5 | 36.3 | 35.7 | 21.7 | 26.8 |
| | Range | (553-1456) | (1145-2793) | (1296-5318) | (1098-3379) | (1586-2853) | (579-1435) |
| t_{1/2} | Mean ± SD | 6.4 ± 3.1 | 5.9 ± 2.1 | 6.4 ± 1.4 | 6.3 ± 3.1 | 6.3 ± 1.5 | 4.8 ± 1.4 |
| (h) | % CV | 49.0 | 35.3 | 21.9 | 49.6 | 24.5 | 30.0 |
| | Range | (3.5-12.4) | (3.3-10.0) | (3.8-7.9) | (1.6-11.9) | (4.1-8.6) | (3.3-7.7) |
| AUC | Mean ± SD | 19.7 ± 7.5 | 24.8 ± 9.5 | 27.4 ± 10.5 | 30.0 ± 14.1 | 55.6 ± 20.5 | 12.2 ± 3.6 |
| Metabolic | % CV | 38.2 | 38.2 | 64.9 | 47.0 | 36.9 | 29.2 |
| Ratio | Range | (12.5-33.8) | (11.2-40.9) | (13.8-50.6) | (10.5-54.2) | (28.7-92.7) | (5.6-17.0) |

¹AUC M-3-G/AUC Morphine

Based on the 95% CI criteria dose proportionality could not be concluded for morphine-3-glucuronide using Cₘₐₓ, AUCₜ and AUC for the morphine formulation. As with morphine-6-glucuronide shorter tₘₐₓ ranges and median values for oral and iv treatments were observed compared to intranasal treatments. Mean half-life times were longer than those observed for morphine and morphine-6-glucuronide. AUC and AUCₜ from the formulation were found to be statistically significantly higher compared with the results from the intravenous formulation. Similar to morphine-6-glucuronide, statistically significantly lower Cₘₐₓ, values were obtained from all dose levels of the morphine formulation compared with the oral formulation. As expected, the formation of M-3-G from morphine was greatest following oral morphine sulphate due to first pass metabolism and least after intravenous infusion of morphine sulphate. In general the metabolic ratios following the intranasal formulations were comparable ranging between 24.8 and 30.0, again somewhere between the two values for oral and intravenous infusion. The metabolic ratio for M-3-G was greater than that for M-6-G regardless of the route of administration.

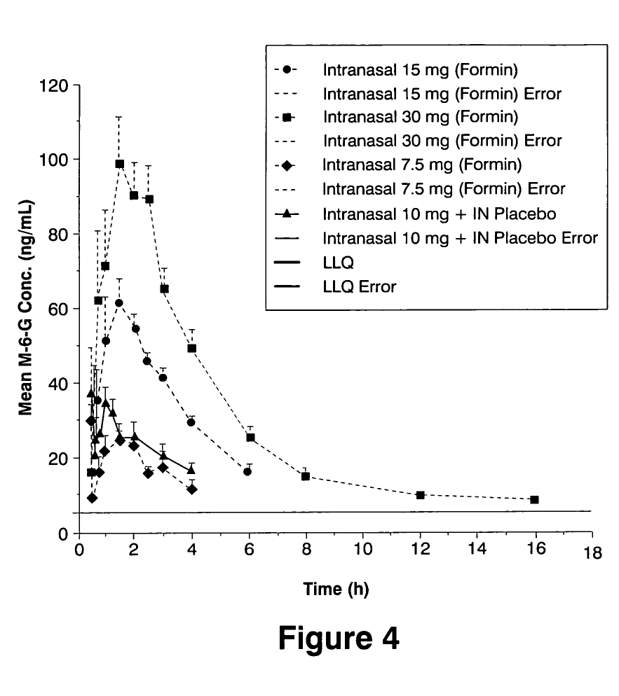

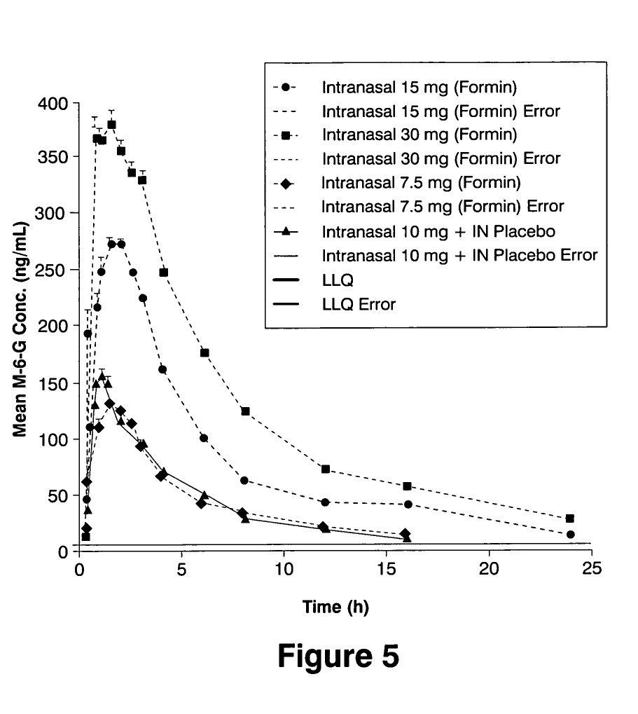

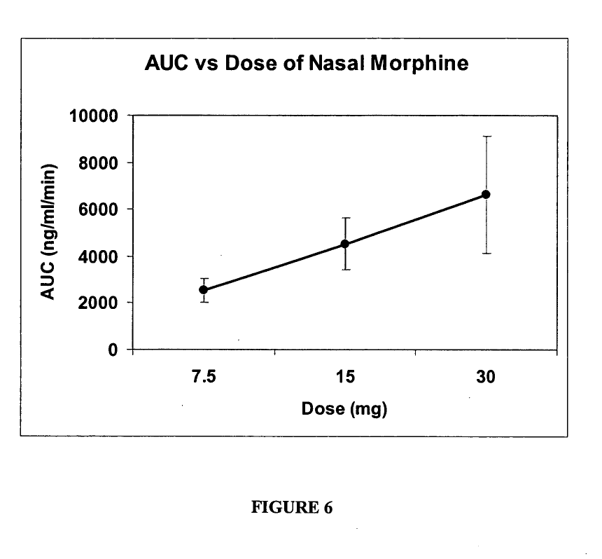

The metabolic profile of intranasal morphine is similar to that of morphine delivered by intravenous infusion as indicated in FIGS. 4 (M-6-G) and 5 (M-3-G). Also, analgesic levels of morphine can be attained within five minutes following nasal administration. In addition, there is a linear relationship between the bioavailability and dose delivered as measured by area under the curves (AUC). See FIG. 6. This observation strongly suggests that the chitosan facilitates the absorption of morphine transmucosally in a dose-dependent fashion.

### CONCLUSIONS

Intranasal tolerability of the morphine formulation for single doses was generally good. Following formulation doses there were 16 reports of nasal symptoms above a rating score of 8; 7.5 mg (3), 15 mg (8) and 30 mg (5). The majority of symptom reports were made at 5 and 15 minutes post-dose, with few symptoms reported post 1 hour after administration. Overall, taste disturbance and sore and stinging nose were the most common symptoms.
Intranasal tolerability of morphine alone (Contains no chitosan) for single doses was generally good. Following 15 mg, there were 2 reports of nasal symptoms above a rating score of 8. The majority of symptom reports were made at 5 and 15 minutes post-dose, with few symptoms reported post 1 hour after administration. The most common symptoms reported were taste disturbance and dry stuffy nose.
Intranasal placebo administration was extremely well tolerated, with only two symptom reports of subjective rating one made. Both related to taste disturbance, no sneezing occurrences were reported.
Absolute bioavailability of morphine from the morphine formulation relative to intravenous dosing was found to be 82.3%, 74.9% and 60.4% for doses of 7.5 mg, 15 mg and 30 mg, respectively.
The increases in Cₘₐₓ, AUCₜ and AUC for morphine, M-6-G and M-3-G were not found to be statistically significantly dose-proportional.
Morphine bioavailability after the morphine formulation compared to morphine alone (no chitosan) treatments was found to be 139.8%, 127.1% and 102.5% for doses of 7.5 mg, 15 mg and 30 mg, respectively.
Relative bioavailability of morphine after the morphine formulation relative to oral morphine sulphate based on AUC values was found to be 218.2%, 198.5% and 160.1% for the 3 dose levels, respectively.
The formation of M-6-G and M-3-G from morphine was greatest following oral morphine sulphate, least following intravenous morphine sulphate and in-between following intranasal administration.

This data, taken together, suggest that chitosan acts to mediate release of morphine to the bloodstream through the nasal mucosa in a regularized fashion suggesting that chitosan acts to mediate controlled absorption.

This unique observation may be attributable to the formulation and potentially more broadly to chitosan containing formulations in general. To date, the only properties that have been published regarding the mechanism of action underlying the activity of chitosan has been related to increasing the residence time of orally or nasally administered drugs to mucosal membranes based on adhesive properties (reviewed by Harding, SE.; Biochem Soc. Trans. 2003, Oct. 31 (Pt.5), 1036-41. The molecular processes underpinning such “mucoadhesive” phenomena have not been elucidated. Given that the data demonstrate that chitosan can act to mediate absorption of drugs such as morphine in a stoichemetric fashion suggests that there are specific mechanisms involved. Most importantly and based on the data, it demonstrated, that pharmaceutical preparations can be made that enable delivery of drug with predictability and therefore safely.

### EXAMPLE 6

### Safety, Tolerability, and Pharmacokinetic Profile of Intranasal Morphine Formulations

This example presents a double-blind, single- and multiple-dose study to assess the safety, tolerability, and pharmacokinetic profile of three ascending dose levels of an intranasal controlled release morphine and chitosan solution in healthy subjects.

The objectives of this study were to examine and compare the single- and multiple-dose safety and tolerability of three dose levels of the morphine formulation with respect to intranasal placebo (saline solution) and to determine and compare the single- and multiple-dose pharmacokinetic profiles of three dose levels of formulation.

Thirty-six healthy male and female subjects were planned to be enrolled into the study. Forty-eight subjects were included in safety and tolerability analyses, and 25 subjects were included in the pharmacokinetic analyses.

This study was originally planned for 36 subjects to be assigned to 3 cohorts. However, due to incorrect dosing and subsequent premature withdrawal of all 12 subjects in the first cohort, an additional 12 subjects were enrolled into this study to replace the first 12 subjects, resulting in a total of 48 subjects. All 12 subjects who were dosed incorrectly (15 mg rather than 7.5 mg) received 3 days of dosing with study medication before being withdrawn. Therefore, all available safety data, nasal examination data, and nasal symptom scores from these subjects were summarized and presented in this study report.

Healthy male and female subjects between the ages of 18 and 60 with no structural or functional abnormalities of the nose and upper airway, obstruction of the nasal passages, or mucosal lesions of the nostrils.

The drug vehicle contains chitosan glutamate, methanesulfonic acid, edetate sodium, benzalkonium chloride, and water. An aqueous nasal spray composition is prepared from the following components:

| Formula concentration: | Conc. 1 | Conc. 2 | Conc. 3 |
|---|---|---|---|
| Ingredients | Weight/ml | Weight/ml | Weight/ml |
| Morphine, anhydrous base | 37.5 mg | 75.0 mg | 150 mg |
| Methanesulfonic acid | 12.7 mg | 25.3 mg | 50.6 mg |
| Benzalkonium chloride | 0.15 mg | 0.15 mg | 0.15 mg |
| (BAK) | | | |
| Edatate Disodium, USP | 1.0 mg | 1.0 mg | 1.0 mg |
| Chitosan | 5.0 mg | 5.0 mg | 5.0 mg |
| WFI Water | QS to 1 ml | QS to 1 ml | QS to 1 ml |
| Molecule Ratio of | ˜11,500:1 | ˜23,000:1 | ˜46,000:1 |
| Morphine:Chitosan | | | |

The test product, dose and mode of administration, and duration of treatment were as follows:
7.5 mg dose level: 3.75 mg of morphine in 100 μL of vehicle, one spray per nostril.
15 mg dose level: 7.5 mg of morphine in 100 μL of vehicle, one spray per nostril.
30 mg dose level: 15 mg of morphine in 100 μL of vehicle, one spray per nostril.

Subjects received a single dose of study medication on Days 1 and 7 and were dosed every six hours on Days 2 through 6. Naltrexone was administered daily to block development of unpleasant effects and tolerance to morphine and the potential for withdrawal effects at the end of the study.

Criteria for evaluation include the pharmacokinetics, tolerability, and safety as follows.

Pharmacokinetics: Blood samples were collected pre-dose and at 5, 10, 15, 30, and 45 minutes and at 1, 1.25, 1.5, 2, 3, 4, 6, 8, 12, 16, and 24 hours after the morning dose of study medication on Day 1 and Day 7 for pharmacokinetic analyses. Blood samples were also collected 15 minutes before the morning dose of study medication on Days 3, 4, 5, and 6.

Tolerability: Tolerability was measured by nasal examinations (measuring severity of rhinorrhea, mucosal erythema, bleeding, and residue) performed on Days 1, 2, 3, 5, and 7, and nasal symptom scores recorded using a 100 mm visual analog scale on Days 1, 2, 3, 5, and 7.

Safety: Safety variables included adverse events, vital signs, and laboratory assessments.

Plasma levels of morphine and its metabolites were tabulated and summarized for individual subjects. The following pharmacokinetic parameters were calculated for single and multiple dose regimens of morphine using a validated pharmacokinetic analysis program: Cₘₐₓ, Tₘₐₓ, t½, AUC, and dose proportionality. Additional analyses for morphine and/or its metabolites were performed as the data allowed.

Continuous variables were presented using summary statistics including number of non-missing observations, mean, standard deviation, median, maximum, and minimum. Categorical variables were summarized using frequency counts and percentages. All collected data was presented in subject listings. No formal statistical tests were performed on the clinical and safety assessments. Results of the nasal examination for rhinorrhea, mucosal erythema, bleeding, and residue were converted to a numerical ordinal scale and summarized using the number of non-missing observations, mean, standard deviation, and median. The nasal symptom scores (using a 100 mm visual analog scale) were summarized using the number of non-missing observations, mean, standard deviation, and median.

Vital signs were summarized using the number of non-missing observations, mean, standard deviation, and median. Clinical laboratory evaluations for which the results were continuous were summarized using the number of non-missing observations, mean, standard deviation, median, minimum, and maximum. All adverse events were tabulated by COSTART body system, COSTART preferred term, and treatment. A frequency bar chart of the proportions of subjects in each treatment experiencing an adverse event was presented by study day of the start of the adverse event. Separate bar charts were generated to present all adverse events and adverse events related to study drug administration.

### Results

Pharmacokinetics: Subjects receiving the morphine formulation intranasally exhibited rapid absorption, with detectable plasma concentrations achieved within five minutes of administration. Steady state conditions were reached within 2 days when the morphine formulation was administered every six hours on Days 2 through 6. The maximum plasma concentration (Cmax) and area under the curve (AUC) were reasonably proportional to dose. Mean values for Cmax on Day 7 were comparable to those on Day 1 in all dosing groups, indicating no accumulation. Mean values for AUC_{∞} on Day 1 were similar to those for AUCss on Day 7, implying linearity in the pharmacokinetics of morphine within a given dose. Mean half-lives (t½) ranged from 2 hours to 11 hours on Day 1 and from 9 to 10 hours on Day 7. The pharmacokinetics of morphine-6-glucuronide (M6G) and morphine-3-glucuronide (M3G) were consistent with those of morphine. Mean plasma concentrations increased proportionally to the increase in dose on Day 1 and Day 7 and were ˜2-fold higher on Day 7 than on Day 1 for all 3 doses. Mean values for AUC_{∞} on Day 1 were comparable to those for AUCss on Day 7, suggesting linearity in the pharmacokinetics of both glucuronide metabolites. Mean t½s for M6G ranged from 2 hours to 9 hours on Day 1 and from 10 to 11 hours on Day 7 and those for M3G from 7.6 hours to 9.5 hours on Day 1 and from 8.7 to 11 hours on Day 7.

Tolerability: For nasal examinations, the majority of rhinorrhea, mucosal erythema, bleeding, and residue observed were mild and did not increase in severity after repeated dosing. The occurrences of rhinorrhea, mucosal erythema, bleeding, and residue in the formulation groups (30 mg, 15 mg, and 7.5 mg) were comparable to the placebo group. For the nasal symptom scores, the majority of the subjects recorded low scores on the VAS for symptoms of runny nose, sore nose, itchy nose, stuffy nose, dry nose, sore throat, and abnormal taste. Of the subjects experiencing runny nose, sore nose, itchy nose, stuffy nose, dry nose, sore throat, and abnormal taste, most of the occurrences were rated less than 50 mm on the VAS. Nasal symptoms did not increase in severity after repeated dosing.

Safety: Treatment-emergent AEs occurred in 8 subjects in the 30 mg group (89%), 18 subjects in the 15 mg group (100%), 8 subjects in the 7.5 mg group (89%), and 9 subjects in the placebo group (75%). The most common treatment-emergent AEs were rhinitis (56% of 30 mg subjects, 78% of 15 mg subjects, 56% of 7.5 mg subjects, and 17% of placebo subjects), taste perverse (44% of 30 mg subjects, 67% of 15 mg subjects, 11% of 7.5 mg subjects, and 0% of placebo subjects), pharyngitis (56% of 30 mg subjects, 44% of 15 mg subjects, 0% of 7.5 mg subjects, and 0% of placebo subjects), headache (11% of 30 mg subjects, 44% of 15 mg subjects, 22% of 7.5 mg subjects, and 17% of placebo subjects), and nausea (11% of 30 mg subjects, 33% of 15 mg subjects, 22% of 7.5 mg subjects, and 25% of placebo subjects). Of the most commonly occurring AEs (rhinitis, taste perverse, pharyngitis, headache, and nausea), all were considered related to study drug. The majority of the AEs reported were mild in severity and decreased in frequency and severity over seven days of repeated administration (up to 22 exposures per subject).

Severe adverse events were reported by three patients, vomit (6% in the 15 mg group) and rhinitis (6% in the 15 mg group and 11% in the 7.5 mg group). There were no noteworthy laboratory values noted on Day 8 or at study exit. No noteworthy changes in blood pressure, pulse, or respiratory rate were recorded during the study. The most common abnormal screening physical examination findings were in the skin system (40 occurrences; 89% of subjects in the 30 mg and 15 mg groups, 78% of the 7.5 mg group, and 75% of the placebo group) and in the mouth/throat/neck system (23 occurrences; 67% of the 30 mg group, 44% of the 15 mg group, 56% of the 7.5 mg group, and 33% of the placebo group).

### CONCLUSION

The results of this study demonstrate that repeated dosing with self-administered intranasal morphine in the formulated vehicle is safe and well tolerated by male and female healthy volunteers. Pharmacokinetic results showed that the formulation was rapidly absorbed and achieved detectable concentrations in plasma within five minutes.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, procedures, and the like are cited throughout this application and in the bibliography, the disclosures of which are incorporated herein by reference in their entireties.

1. An aqueous transmucosally delivered controlled release composition which upon administration exhibits linear absorption rates, the composition comprising:
(a) a therapeutically effective amount of a pharmaceutically active ingredient;
(b) an effective amount of a controlled release chitosan polymer;
and optionally comprising:
(c) one or more antimicrobial agents;
(d) one or more antioxidants; and
(e) water;
wherein the molecule to molecule ratio of the pharmaceutically active ingredient to the controlled release chitosan polymer ranges from about 1:1 to about 100,000:1.

2. The composition of claim 1, wherein the molecule to molecule ratio of the pharmaceutically active ingredient to the controlled release chitosan polymer ranges from about 5,000:1 to about 80,000:1.

3. The composition of claim 1, wherein the pharmaceutically active ingredient is morphine.

4. The composition of claim 3, wherein the concentration of morphine is from about 18.75 mg/ml to about 300 mg/ml.

5. The composition of claim 3, wherein the concentration of morphine is from about 37.5 mg/ml to about 150 mg/ml.

6. The composition of claim 3, wherein morphine is purified morphine base monohydrate.

7. The composition of claim 1, wherein the concentration of the chitosan polymer is from about 2 mg/ml to about 7 mg/ml.

8. The composition of claim 1, wherein the concentration of the chitosan polymer is from about 4 mg/ml to about 6 mg/ml.

9. The composition of claim 1 wherein the antioxidant is selected from the group consisting of methanesulfonic acid, citric acid, sodium citrate, ascorbic acid, and sodium ascorbate.

10. The composition of claim 9, wherein the antioxidants are citric acid and sodium citrate, and the total amount of antioxidant is present in a range from about 20 to about 50% by weight/volume of the composition.

11. The composition of claim 9, wherein the antioxidants are ascorbic acid and sodium ascorbate, and the total amount of antioxidant is present in a range from about 40 to about 70% by weight/volume of the composition.

12. The composition of claim 9, wherein the antioxidant is methanesulfonic acid, and the amount of antioxidant is present in a range from about 10 to about 60% by weight/volume of the composition.

13. The composition of claim 1, wherein the antimicrobial agent is selected from the group consisting of benzalkonium chloride, disodium EDTA, sodium benzoate, and combinations thereof.

14. The composition of claim 12, wherein the concentration of antimicrobial agent is from about 0.0005% to about 0.5% by weight/volume of the composition.

15. The composition of claim 12, wherein the concentration of antimicrobial agent is from about 0.005% to about 0.5% by weight/volume of the composition.

16. The composition of claim 1, wherein the transmucosal delivery is selected from the group consisting of nasal, buccal, rectal, vaginal, and ocular modes of administration.

17. The composition of claim 1, wherein the transmucosal delivery is by nasal administration.

18. The composition of claim 1, wherein the composition is prepared under nitrogen gas by
(a) mixing the morphine and acid, polymer, and antimicrobial agents, wherein each ingredient is mixed into the solution for at least 5 minutes;
(b) adding the antioxidants, wherein the pH is from about 3.0 to about 5.0;
(c) adjusting the final batch volume with water to form a final solution; and
(d) filtering the solution with a pre-sterilized micron filter.

19. The composition of claim 18, wherein the pre-sterilized micron filter is about a 0.2 micron filter.

20. The composition of claim 1, wherein the composition yields about 18.75 to about 300 microgram of pharmaceutically effective agent per 100 microliter nasal spray.

21. A method of administering an aqueous controlled release transmucosal medicament, wherein the medicament is administered transmucosally to a subject in need thereof, said medicament comprising:
(a) a therapeutically effective amount of a pharmaceutically active ingredient;
(b) an effective amount of a controlled release chitosan polymer; and optionally comprising:
(c) one or more antimicrobial agents;
(d) one or more antioxidants; and
(e) water.

22. The method of claim 21, wherein the pharmaceutically active ingredient is purified morphine base monohydrate.

23. The method of claim 21, wherein the subject is human.
